(19) United States
(12) Patent Application Publication
(10) Pub. No.: US 2007/0032842 A1
(43) Pub. Date: Feb. 8, 2007

(54) Phototherapy Device for Treating Hyperbilirubinemia

(76) Inventor: Nathan Rulon Strong, Sandy, UT (US)

Correspondence Address: Nathan Rulon Strong, 8638 S. 910 E., Sandy, UT, 84094, US

(21) Appl. No.: 11/161,446

(22) Filed: Aug. 3, 2005

(51) Int. Cl. A61N 5/06

(52) U.S. Cl. 607/88

(57) ABSTRACT

Jaundice, also known as hyperbilirubinemia, is a common condition experienced by newborn babies. In severe cases it is treated by intense phototherapy. Currently, special halogen lights and fiber optics are used to expose the baby's skin to blue light, which transforms bilirubin into more water-soluble forms that can be easily eliminated from the body. The problem is that these phototherapy devices are often cumbersome, uncomfortable, and expensive. They are impractical to own because of their expense and short term of use. The present invention is a phototherapy flashlight device that uses a powerful light-emitting diode (LED), to produce a specific wavelength of blue light. This bilirubin flashlight may be held by hand or positioned within a support structure to shine on a fixed area. This invention is new and novel because it is not obvious to replace the more desirable white light of a common flashlight with an LED that emits a specific wavelength of blue light in the range of 400 nm-500 nm at an intensity that transforms bilirubin into more water-soluble forms. Its advantages over prior art are that it is fairly cheap, readily available, and easy to use. It is also portable and can be used at convenient times and places. Such a device could be purchased by the average consumer and will be very effective at treating jaundice.

### FIELD OF THE INVENTION

This invention is a phototherapy device for treating jaundice, which is also known as hyperbilirubinemia. The invention emits a particular wavelength of blue light that is suitable for the conformational and structural isomerization of bilirubin into water-soluble forms that are easier for the body to eliminate. This device is similar to a flashlight wherein the light-emitting portion is comprised of one or more powerful light-emitting diodes (LEDs) that emit a specific wavelength of blue light.

### BACKGROUND OF THE INVENTION

Jaundice is a condition experienced by many newborn babies where abnormally high levels of bilirubin have accumulated in their body. Its most pronounced symptom is yellow coloring of the skin and eyes. Jaundice is also known as hyperbilirubinemia.

Bilirubin is primarily a byproduct of the death and decomposition of red blood cells. Normally, bilirubin is conjugated with glucuronic acid in the liver so that it can be eliminated from the body through the bile. However, several of the proteins and enzymes that perform this function are not present at the necessary levels in newborns. This leads to a rise in bilirubin levels in their blood. Since the bilirubin is not water-soluble it tends to accumulate in body tissues, thereby causing the yellow coloration. The danger of high levels of bilirubin is that it can be neurotoxic. If levels get too high there can be nerve damage, or even death.

In most cases, jaundice is not very severe and disappears within a few weeks as the baby's body develops to a point where it is better able to rid itself of the bilirubin. However, if bilirubin levels are too high when the baby is still very young, then treatment may be necessary to prevent possible nerve damage. The most common treatment for jaundice is phototherapy.

Phototherapy is comprised of shining light onto skin. The light penetrates the skin to a certain degree and interacts with bilirubin. Bilirubin has the chemical structure shown in FIG. 1A. The carboxyl groups form hydrogen bonds with nearby nitrogen atoms, which hides the hydrophilic moieties and increases the molecule's overall hydrophobicity. Bilirubin's hydrophobicity makes it dangerous because it will readily absorb into tissue.

Phototherapy light can cause two distinct and important changes of bilirubin. The first is a configurational isomerization where intramolecular hydrogen bonds within bilirubin are broken. This change exposes a carboxyl group and helps makes bilirubin water-soluble for a limited time. Water-soluble bilirubin is safer because it cannot easily enter tissues or penetrate the blood-brain barrier; it is slowly eliminated through the bile. In this manner, phototherapy reduces the amount of total bilirubin that can potentially cause damage by about 20% during phototherapy (Hansen, TWR; “Neonatal Jaundice”; www.emedicine.com/ped/topic1061.htm; 2002). The second change is a structural isomerization of bilirubin to lumirubin. This change is irreversible and exposes one of the carboxyl groups, thereby making lumirubin more hydrophilic than bilirubin. The liver easily eliminates lumirubin through the bile. The chemical structure of lumirubin is shown in FIG. 1B. The amount of lumirubin produced by phototherapy is dependant on the intensity of the light. Higher light intensities generate more lumirubin. The quick elimination of lumirubin may account for the majority of total serum bilirubin reduction provided by phototherapy (Costarino, AT et al; Pediatrics; 75 (3), 519-522,1985)

Phototherapy is considered to be extremely safe. The only potential risk is damage to a baby's eyes by the intense light. Therefore, it is highly recommended that the baby's eyes be covered appropriately during phototherapy. Otherwise, no significant side effects of phototherapy have been documented.

The appropriate wavelength of light varies within the available literature. As described in the “Neonatal Jaundice” article from the eMedicine.com website cited in paragraph 6, the most appropriate wavelength for treating jaundice is 450 nm, which is the most efficient wavelength for the isomerization of bilirubin. It is likely that wavelengths that fall within the range of 400 nm -500 nm, and more specifically 445 nm -475 nm, will have a beneficial effect. In addition, a light intensity of at least 6 microWatts per square centimeter per nanonmeter of light wavelength is also needed. This intensity is equivalent to 2.7 milliwatts per square centimeter of 450 nm wavelength light. An article by Daniel Seidman, et al. suggests that higher wavelengths may be better because the higher wavelengths penetrate deeper through skin, thereby allowing more interaction with bilirubin (Seidman, D, et al; Journal of Perinatology; 23, 123-127, 2003).

The prior art suggests several ways to illuminate skin with blue light. Most commonly halogen lights, or even fiber optics, are used to direct blue light at a baby's skin. In recent instances light-emitting diodes (LEDs) have been used in arrays implanted in clothing or otherwise near the baby to illuminate its skin with blue light. One example of a study conducted using an array of LEDs is found in a paper by Yun Sil Chang (Chang, YS, et al; J. Korean Med. Sci; 20, 61-64, 2005). The advantage of using LEDs is that they emit a narrow spectrum of light so that no filtering is required. Also, LEDs do not heat up to the point of causing physical damage to skin. Typically, phototherapy devices have been designed primarily for use in hospitals and some limited use at home.

No prior art teaches that a modified LED flashlight, that emits specific wavelength blue light, may serve as a phototherapy device for the treatment of hyperbilirubinemia. One of the reasons may be that LEDs have not been powerful enough in the past to serve as an effective phototherapy unit within a flashlight. With regard to specific wavelength flashlights, there are current US patent applications, document number 20020074559 by K. J. Dowling and document number 20040176824 by M. V. Weckwerth, which introduce flashlights using UV wavelengths. These devices would not be suitable for the treatment of jaundice because of skin damaging UV, nor do they teach that blue light wavelengths may be used for treating hyperbilirubinemia.

The bilirubin flashlight disclosed herein fulfills a need for a low-cost, portable device that may be used to treat hyperbilirubinemia of various severities. The small nature of the bilirubin flashlight makes it convenient for carrying in a diaper bag for use almost anywhere. It is very convenient because it can be easily directed toward any part of the body, including exposed arms and legs. This allows the baby to stay mostly dressed, thereby being warm and comfortable during treatment. This is a direct improvement over typical devices that require that a baby be stripped of clothing down to his/her diaper, which can be cold and uncomfortable for the baby.

### BRIEF SUMMARY OF THE INVENTION

This invention modifies a flashlight with one or more light-emitting diodes (LEDs) to emit a specific frequency of blue light. The bilirubin flashlight can be either hand-held or fixed in position with a support structure. Also, it may have embodiments that use a power source of DC batteries and/or household AC current.

The term “flashlight” shall be defined herein to describe any portable, light-emitting device that emits light in a particular direction, either diffuse or in a beam. It is recognized that flashlights come in many shapes and sizes, so no dimensional restrictions will be mentioned herein because it is inconsequential to the effectiveness of the device. The most basic elements necessary to constitute a flashlight are a power source (either DC or AC), structural housing (of any shape, composition, or size), means for emitting light, and means for guiding/reflecting the light in a particular direction (such as collimating optics, reflectors, and lenses).

The primary function of the bilirubin flashlight device is to illuminate skin with blue light in order to cause structural and conformational isomerization of bilirubin. It will most often be used to treat jaundice in newborns, but theoretically may be used on a person of any age.

There are many cases where a baby has elevated bilirubin levels that are not high enough to justify the cost and inconvenience of present phototherapy devices or hospitalization. The present invention will provide a cost-effective alternative to current phototherapy techniques. It will be cheaper, readily available, easy to use, and may be prescribed for intermediate cases where intense phototherapy is not essential; in this case, it will be helpful in alleviating the mother's worrying.

### BRIEF DESCRIPTION OF THE DRAWINGS

FIG. 1. A) Chemical structure of bilirubin. B) Chemical structure of lumirubin.

FIG. 2. One embodiment of the bilirubin flashlight comprised of one or more LEDs (1), and a flashlight housing (2).

FIG. 3. A second embodiment of the bilirubin flashlight that also uses one or more LEDs (1), and a different flashlight housing style (3).

FIG. 4. The bilirubin flashlight, held by hand, shining on the skin of an infant to treat hyperbilirubinemia.

FIG. 5. The bilirubin flashlight shining on the skin of an infant where the flashlight is being held in place by a fixed structure (4), rather than being held by hand.

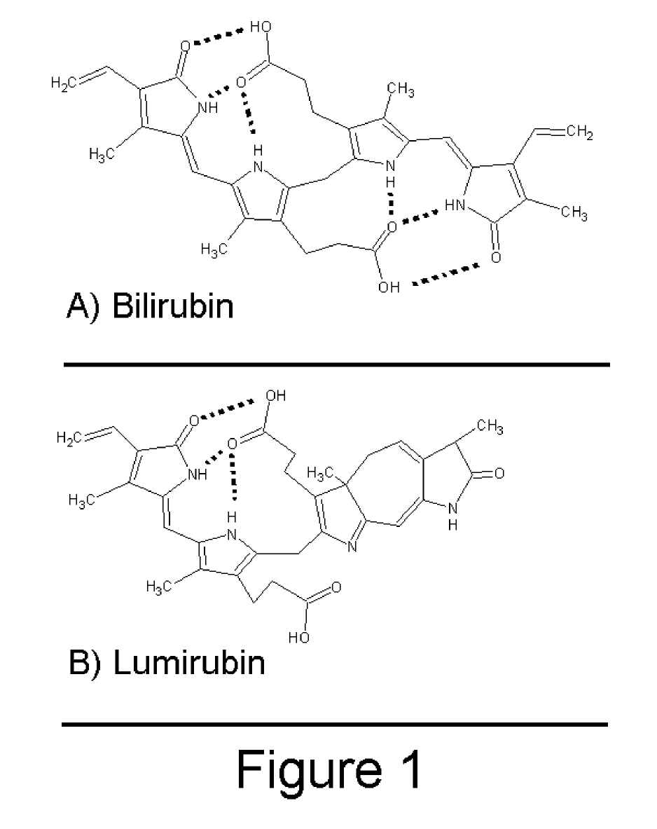

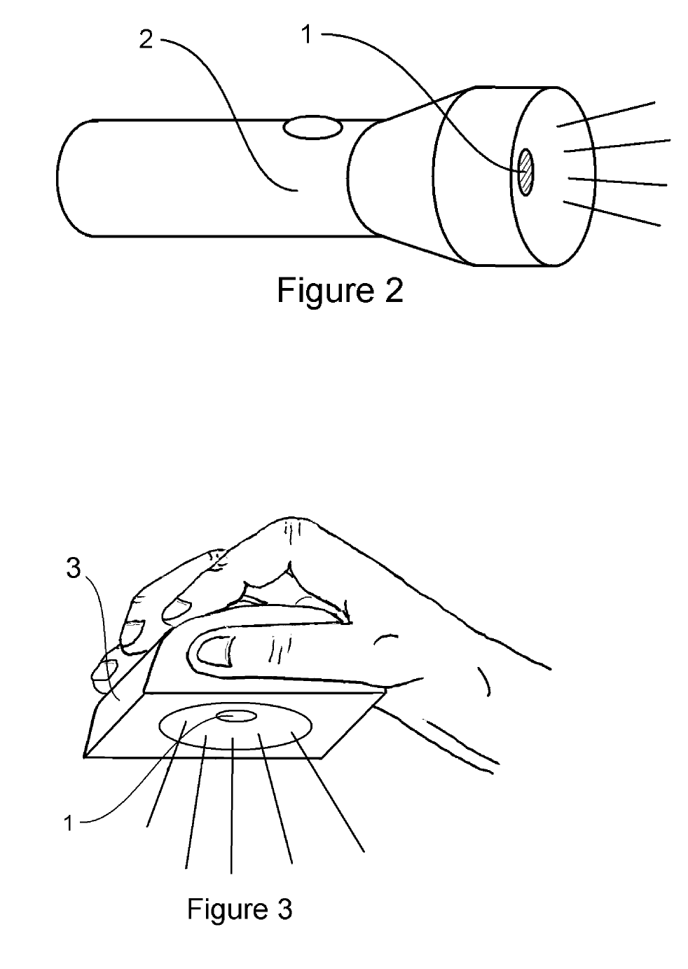

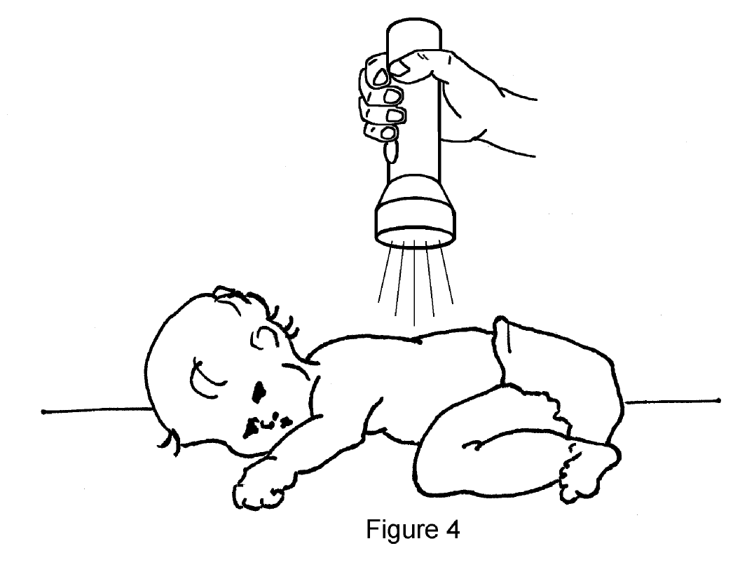

### DETAILED DESCRIPTION OF THE INVENTION

This invention is made by replacing the light bulb of a flashlight with one or more LEDs that emit blue light with a wavelength in the range of 400-500 nm. Appropriate electrical changes may be made in series with the power source to provide the appropriate voltage and current to power the LED(s). The changes in the power system will depend on the specifications of the particular LED used. Typical flashlight components, like various housing styles, reflectors, collimating optics, or lenses may be used. A high intensity light-emitting diode is required to treat hyperbilirubinemia. In practice any type of LED could be used as long as it gives off enough light. Luxeon® LEDs by Lumileds™ are one example of an LED that produces light with an adequate intensity.

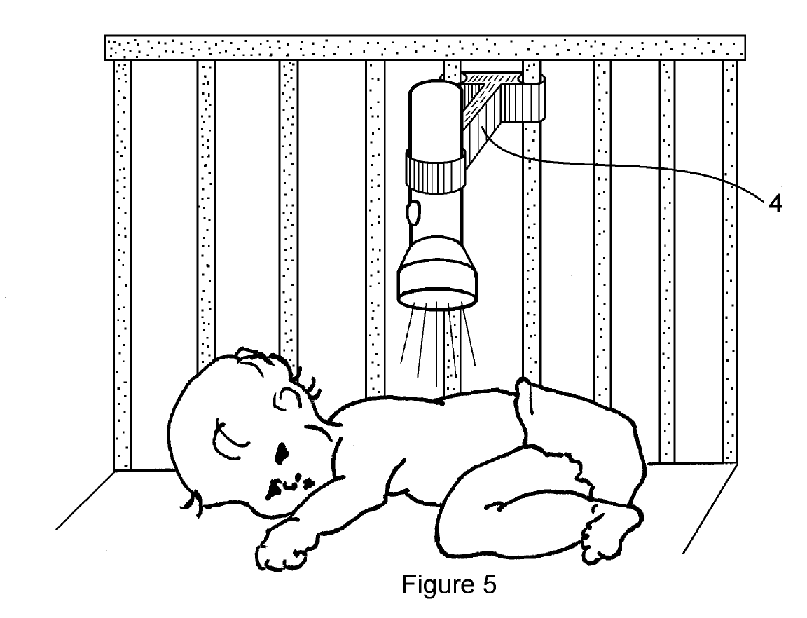

The most common method for using this bilirubin flashlight will be to lift or remove a baby's clothing and then shine the light on the baby's exposed skin for as long as possible as shown in FIG. 4. This may be done while the baby is sleeping, nursing, feeding, or awake. The person shining the flashlight on the baby may hold the flashlight by hand or place the flashlight in a holding structure to shine on one area continuously as shown in FIG. 5. While shining the light on the front of the baby, it is advised to cover the baby's eyes to prevent possible damage. However, the nature of the flashlight will usually provide a beam spot on the baby's skin that can be easily directed away from the eyes to prevent damage. Another advantage of using a highly portable light device is that it can be easily moved to shine on multiple areas of the baby, thereby illuminating more skin and converting more bilirubin to lumirubin. It is also not necessary to undress the baby completely because the light may be shined on exposed arms or legs.

Any type of physical structure may be used to hold the bilirubin flashlight in place so that it shines on one area of skin continuously. Or, a structure in motion may be used to automatically move the bilirubin flashlight to shine on different areas. The structure can be rigid or flexible as long as it has a means for holding the bilirubin flashlight so that it illuminates a particular area. A flexible structure, or one with multiple adjustment points, would provide ease to the user when trying to adjust the light to shine on a particular location. Also, certain coverings around the lens may help guide or reflect the light to a desired location. Any type of light guide, reflector, or collimator could be used in this instance.

The present invention is new and novel. It is not obvious over the prior art. The main purpose of flashlights has traditionally been to provide illumination for seeing things in the dark, and they most commonly utilize white light. It is not obvious to replace a flashlight's means for producing white light with means for producing blue light that specifically has a wavelength around 450 nm. Furthermore, it is not obvious to use said flashlight, which produces blue light, as an instrument for treating hyperbilirubinemia. From the perspective of flashlight prior art, it is not obvious to one skilled in flashlight-making to replace a white light with a blue light around 450 nm wavelength.

The bilirubin flashlight described herein fulfills a need in the phototherapy market for a cheap, convenient, easy to use, and portable phototherapy device.

What is claimed is:

1. A phototherapy flashlight device that utilizes one or more LEDs to produce blue light with a wavelength between 400-500 nm and is used to treat hyperbilirubinemia.

2. A method of using the device of claim 1, wherein the device is held by hand and used to illuminate skin in order to treat hyperbilirubinemia.

3. A method of using the device of claim 1, wherein the device is held in a fixed position by a holder, stand, grip, frame, rope, string, chain or other rigid or flexible structure, so as to illuminate a particular area in order to treat hyperbilirubinemia without the need to hold the device by hand.
